(19) United States
(12) Patent Application Publication
(10) Pub. No.: US 2022/0257165 A1
(43) Pub. Date: Aug. 18, 2022

(54) Step Counting System and Method

(71) Applicant: Yongfang Wang, Xi'an City (CN)

(72) Inventor: Yongfang Wang, Xi'an City (CN)

(21) Appl. No.: 17/737,146

(22) Filed: May 5, 2022

Related U.S. Application Data
(63) Continuation-in-part of application No. 17/607,873, filed on Oct. 30, 2021.

(30) Foreign Application Priority Data
May 12, 2019 (CN) 201910391402.1

(51) Int. Cl. A61B 5/242; A61B 5/277; A61B 5/256

(52) U.S. Cl. CPC A61B 5/242; A61B 2562/0223; A61B 5/256; A61B 5/277

(57) ABSTRACT

Disclosed is a step counting method comprising the steps of, firstly, acquiring an electric signal on an electrode that is in contact or coupled with a human skin, the signal is caused by the human body capacitance variation during foot-lifting and foot-landing; secondly, processing the electric signal by a signal processing circuit that is electrically connected with the electrode and outputting a signal stream that records the foot-lifting and foot-landing information to a central control unit; and thirdly, analyzing the received signal stream by the central control unit that is electrically connected with the signal processing circuit to calculate the step number. The proposed step counting method accomplished by monitoring the electric signal on the electrode caused by the human body capacitance variation can improve the step counting accuracy.

### CROSS-REFERENCE TO RELATED APPLICATION

This application claims priority benefits to a U.S. application Ser. No. 17/607,873 filed on Oct. 30, 2021, of a PCT Application #PCT/CN2019/100467 filed on Aug. 14, 2019, which claims priority to a Chinese Application #201910391402.1 filed on May 12, 2019, the contents of which are incorporated herein by reference in its entirety.

### TECHNICAL FIELD

The present disclosure, in some embodiment thereof, relates to the field of electronic communication and technique. More specifically the present disclosure relates to a step counting system and method, utilizing a step counting algorithm via a computer-readable medium.

### BACKGROUND

Nowadays, many people are used to recording their step numbers by smart wristbands, smartwatches, pedometers, and smartphones. By measuring the step numbers, the energy consumption can be estimated to achieve the purpose of health tracking. However, at present, most wearable and portable smart devices with step counting functionality acquire the step numbers via the data collected by the embedded inertial sensor. Due to the complexity of human movement, the accuracy of the step numbers based on the inertial sensor is limited. In essence, the inertial sensor senses the movement of the body part where the sensor is worn. For example, a smartwatch worn on the wrist actually senses the wrist's movement instead of the movement of the leg, which will result in the inaccuracy of step counting. For example, when a wristband wearer is waving his or her hand without walking, the waves of the arm will cause misjudgment of the step numbers. For another example, if a wristband wearer puts his hand in his trouser pocket, the number of walking steps will be challenging to detect. Thus, the inertial sensor-based step counting often fails to measure or mistakenly measures the steps.

Other step counting approaches lack wearing comfortableness since they need extra assistant items for step measurement. For example, the U.S. Pat. No. 11,179,103B2 titled “Wearable step counter system” describes a method of calculating the step numbers based on the parasitic capacitance generated by the relative movement between silk fabric and the leg. However, the approach relies on a particular garment configuration, and massive adoption of the system is limited.

### SUMMARY

The following is the summary of the present disclosure. This summary is not intended to limit the protection scope of the claims.

The first objective of the present disclosure is to provide a novel and accurate step counting method, aiming to overcome the inaccuracy of the traditional inertial sensor-based step counting method.

The second objective of the present disclosure is to provide a novel, low-power, low-cost, and small-form step counting system, which can be embedded into smart devices that need step counting functionality, such as smartwatches, wristbands, and smartphones.

The third objective of the present disclosure is to provide a simple step counting algorithm based on the proposed method and system. The algorithm can be implemented by simple computer instructions. Compared with the traditional inertial sensor-based step counting algorithm, the proposed algorithm is simple, easy to implement, and consumes less energy.

We disclosed a novel step counting system, a step counting method, and corresponding step counting algorithm utilizing a computer-readable storage medium. The steps are counted by monitoring the human body capacitance variation generated electric signal on an electrode, which is in contact or coupled with the human body. The disclosure aims to reduce the probability of failed or false step counting and improve step counting accuracy.

The term “human body capacitance” refers to the electric field between the human body and the environment, especially the ground. The variation of the human body capacitance may also be described as the variation of the electric field between the human body and the environment. The concept of the human body capacitance is established based on the fact that the human body is a good conductor, and a certain amount of charges is distributed around the body. Thus, there is always an electrical field between the human body and the environment, especially the ground. This electric field can be expressed by multiple physical quantities, such as skin potential, human body electrostatic field, human body capacitance, etc. The present disclosure uses human body capacitance to describe this natural electric field. When the human body moves, such as lifting the foot, the distance between the human body and the ground changes, resulting in a variation of the human body capacitance. When the human body capacitance changes, that is, the electric field between the human body and the environment changes, the charges distributed around the human body will flow or redistribute. By directly or indirectly monitoring the variation of human body capacitance, the movement of the human body can be perceived. The foot movement, landing and lifting, has a more significant impact on the human body capacitance. Other non-leg movements, such as arm movements, can also change the human body capacitance, but the impact is relatively small compared to the foot lifting and landing. Thereby accurate step counting can be achieved by monitoring the variation of the human body capacitance.

In a first aspect, the present disclosure provides a step counting method, which comprises the following steps: signal acquiring as the first step, inferring that an electric signal on an electrode that is in contact or coupled with the body occurs caused by the human body capacitance variation when lifting and landing the foot. Signal processing as the second step, inferring that a signal processing circuit processes the electric signal, as described in the first step, and outputs a signal stream that contains the foot-lifting and foot-landing knowledge. Finally, signal analyzing as the third step, inferring that a central control unit that is electrically connected with the output of the signal processing circuit analyzes the received signal stream and counts the step numbers.

The electrode, which is in contact or coupled with the human body, is electrically connected to the input of the signal processing circuit. When the human body capacitance changes, the flow of charges, that is, current, occurs on the electrode. When the electrode is in contact with the skin, the current is a conduction current, and when the electrode is coupled with the skin, the current is a displacement current.

The signal processing circuit has an input impedance larger than one kiloohm and provides a bias voltage to the electrode. This bias voltage also acts as a charge source for the electrode.

According to some embodiments, the signal processing circuit includes, but is not limited to, one or more of the following functions: filtering, amplification, analog-to-digital conversion.

According to some embodiments, the central control unit analyzes the step number by comparing the collected raw data or the change rate of the raw data or the rate of change rate of the raw data with preset thresholds.

In a second aspect, the embodiments of the present disclosure further provide a step counting system, which comprises the following parts: at least one electrode, which is in contact or coupled with human body; a signal processing circuit, whose input terminal is electrically connected to the electrode, providing a bias voltage and supplying charges for the electrode. As a result, the charge flow signal on the electrode can be expressed as the observable and readable voltage variation signal. The signal processing circuit has an input impedance higher than one kiloohm. The signal processing circuit performs signal processing to the electrical signals on the electrode and outputs a signal stream that contains the foot-lifting and foot-landing knowledge; and a central control unit, which is electrically connected to the signal processing circuit, analyzes the signal stream received from the signal processing circuit and calculates step number. During a foot-lifting or foot-landing action, the human body capacitance will change. As a result, a conduction current or displacement current occurs at the electrode. The signal processing unit perceives the current signal and processes it in the form of a voltage signal. After being processed by the signal processing circuit, the signal is sent to and analyzed by the central control unit. Finally, the step number is acquired according to the features of the processed signal.

The system, as mentioned above, processes the charge flow signal on the electrode caused by human body capacitance variation, converts it into a readable voltage signal, and analyzes the step information from the voltage signal.

According to some embodiments, the charge flow signal on the electrodes caused by the human body capacitance variation can also be processed by a circuit that converts it into other electrical signals such as frequency signal, and the step information can be analyzed from the frequency signal.

The term “electrode, which is in contact or coupled with the human skin” refers to a conductor of any shape, size, or material. The electrode is either in direct contact with the skin, physically connecting human skin and signal processing circuit, or coupled with the skin, forming a coupling capacitance, meaning that there is a non-conductor in the middle to isolate the skin and the electrode. In the former case, a conduction current occurs on the electrode during foot-lifting and foot-landing. In the latter case, a displacement current occurs on the electrode during foot-lifting and foot-landing. Since a bias voltage is applied to the electrode, the current signal can be observed as a voltage signal. By performing signal processing on the voltage signal, such as filtering, amplifying, analog-to-digital conversion, the central control unit can obtain the signal caused by the foot lifting and foot landing.

According to some embodiments, The electrode can be a conductor placed inside or on the surface of a smart device; it can also be any conductor integrated into the smart device, such as a conductive layer on a printed circuit board.

According to some embodiments, the signal processing circuit includes, but is not limited to, one or more of the following: signal filtering circuit, signal amplifying circuit, analog-to-digital conversion circuit.

According to some embodiments, in the signal processing circuit, the voltage signal on the electrode is amplified by an amplifier, and the amplified voltage signal is sent to a general analog-to-digital converter, such as an 8-bit analog-to-digital converter.

According to some embodiments, in the signal processing circuit, by placing a high-precision analog-to-digital converter, such as a 24-bit high-precision analog-to-digital converter, the voltage signal on the electrode does not need to be amplified by an amplifier circuit.

In a third aspect, the embodiments of the present disclosure further provide a step counting algorithm corresponding to the step counting method described in the first aspect above and the step counting system described in the second aspect above. The foot-lifting and foot-landing actions cause peak signals in the signal streams obtained by the central control unit. Step numbers can be obtained by counting the peak signals.

Specifically, a first data is obtained from the raw data, which is obtained by acquiring the electrical signal on the electrode that is in contact with or coupled with the human body and performing signal processing on the electrical signal. When the first data meets the preset conditions, obtain the second data. It is also necessary to confirm that the second data meet the preset conditions. Then, the step information can be analyzed by observing the features of the first and second data.

According to some embodiments, the first data and the second data include but are not limited to one or more of the following data: a single raw data acquired at the central control unit, a group of raw data acquired at the central control unit, a single change rate data of the raw data acquired at the central control unit, a group change rate data of raw data acquired at the central control unit, a single rate of change rate data of the raw data acquired at the central control unit, a group rate of change rate data of raw data acquired at the central control unit.

According to some embodiments, the preset condition is a threshold value based on the raw data or the change rate of raw data or the rate of change rate of raw data for comparison.

In a fourth aspect, the embodiments of the present disclosure further provide a computer-readable storage medium, where the computer-readable storage medium stores computer-executable instructions, and the computer-executable instructions are used to cause the computer to execute the step-counting method and algorithm according to the above described aspects.

Other features and advantages of the present disclosure will be elaborated in the following description and will be partially obvious from the description or may be understood from the embodiments of the present disclosure. The objectives and other advantages of the present disclosure may be realized and attained by the description, claims, and appended drawings.

### BRIEF DESCRIPTION OF THE DRAWINGS

The accompanying drawings are used to provide a further understanding of the technical solutions of the present disclosure and constitute a part of the description. They are used to explain the technical solutions of the present disclosure together with the embodiments of the present disclosure and do not constitute a limitation on the technical solutions of the present disclosure.

FIG. 1 schematically shows the human body capacitance model, the electric relation between a human body and the environment;

FIG. 2 schematically shows the electric field variation between the human body and ground when foot-lifting or foot-landing occurs;

FIG. 3 is the basic working steps of a step counting system provided by an embodiment of the present disclosure;

FIG. 4 is a schematic structural diagram of a step counting system provided by an embodiment of the present disclosure;

FIG. 5 is a schematic diagram of a connection mode provided by an embodiment of the present disclosure;

FIG. 6 is a circuit diagram of a signal processing circuit provided by an embodiment of the present disclosure;

FIG. 7 is a circuit diagram of a signal processing circuit provided by another embodiment of the present disclosure;

FIG. 8 is a signal diagram of a step counting method provided by an embodiment of the present disclosure;

FIG. 9 is another signal diagram of a step counting method provided by an embodiment of the present disclosure;

FIG. 10 is a schematic diagram of a system architecture platform for executing a step counting method provided by an embodiment of the present disclosure;

FIG. 11 is a basic flowchart of a step counting algorithm provided by an embodiment of the present disclosure;

FIG. 12 is a flowchart of a part of the step counting algorithm provided by an embodiment of the present disclosure;

FIG. 13 is a flowchart of a part of the step counting algorithm provided by an embodiment of the present disclosure.

### DETAILED DESCRIPTION

To achieve the objectives, technical solutions, and advantages of the present disclosure clearer, the present disclosure will be further described hereinafter with reference to the accompanying drawings and embodiments. It should be understood that the specific embodiments described herein are only used to explain the present disclosure, but are not intended to limit the present disclosure.

In the description of the present disclosure, the meaning of several refers to be one or more, and the meaning of multiple refers to be more than two. The meanings of greater than, less than, more than, etc., are understood as not including this number, while the meanings of above, below, within, etc., are understood as including this number. If there is a description to the first and second, it is only for the purpose of distinguishing technical features, and shall not be understood as indicating or implying relative importance, implicitly indicating the number of the indicated technical features or implicitly indicating the order of the indicated technical features.

In the description of the present disclosure, unless otherwise explicitly defined, words such as setting, installing and connecting should be understood in a broad sense, and those skilled in the art can reasonably determine the specific meanings of the above words in the present disclosure, in combination with the specific contents of the technical solutions.

At present, almost all wearable and portable smart devices with step counting functionality count the steps through the inertial measurement sensor, and the accuracy of the step counting is limited. Due to the complexity of human movement, the step counting based on the inertial measurement sensor often misses to count or mistakenly counts. The identification and calculation of the steps thereof also take up a lot of hardware resources.

To address the above-described problems, the embodiments of the present disclosure provide a novel step counting method, a novel step counting system, an easy-to-implement corresponding step counting algorithm, and a computer-readable storage medium. The method comprises the following steps: signal acquiring as the first step, inferring that an electric signal on an electrode that is in contact or coupled with the body is generated caused by the human body capacitance variation when lifting and landing the foot. Signal processing as the second step, inferring that an electric signal processing circuit processes the electric signal, as described in the first step, and outputs a signal stream that contains the foot-lifting and foot-landing knowledge. Finally, signal analyzing as the third step, inferring that a central control unit that is electrically connected with the output of the electric signal processing circuit analyzes the received signal stream and counts the step numbers.

The embodiments of the present disclosure are further described below with reference to the accompanying drawings.

As shown in FIG. 1: FIG. 1 schematically shows the model of human body capacitance, the electric field between the human body and the surrounding environment. Since the human body is a good conductor, it can store charges. The environment around the human body can be regarded as the ground. So an electric field is formed between the human body and the surrounding environment. The present disclosure describes this electric field in terms of human body capacitance. Human body capacitance is a physical property of the human body itself, and its construction does not depend on external items like garment.

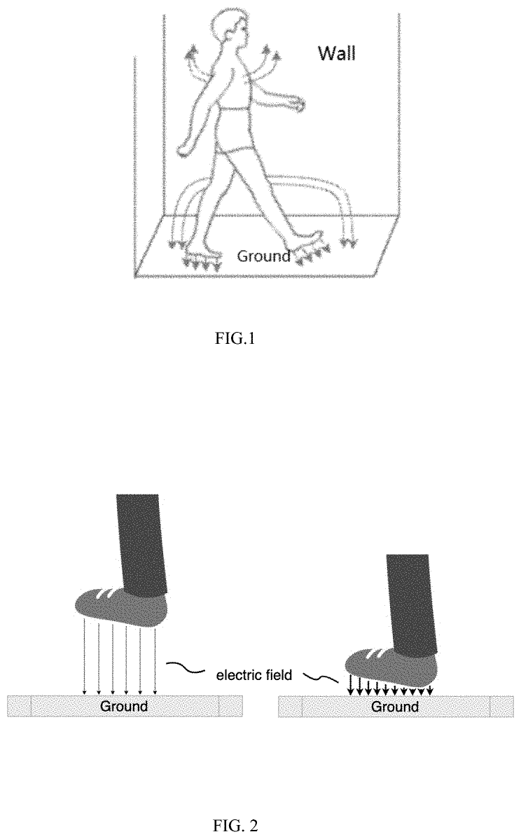

FIG. 2 schematically shows the electrostatic field between the foot and the ground during foot-lifting and foot-landing. As the distance between the foot and the ground changes, the electric field between them also changes. The present disclosure utilizes the phenomenon of human body capacitance variation caused by foot-lifting and foot-landing to perform accurate step counting.

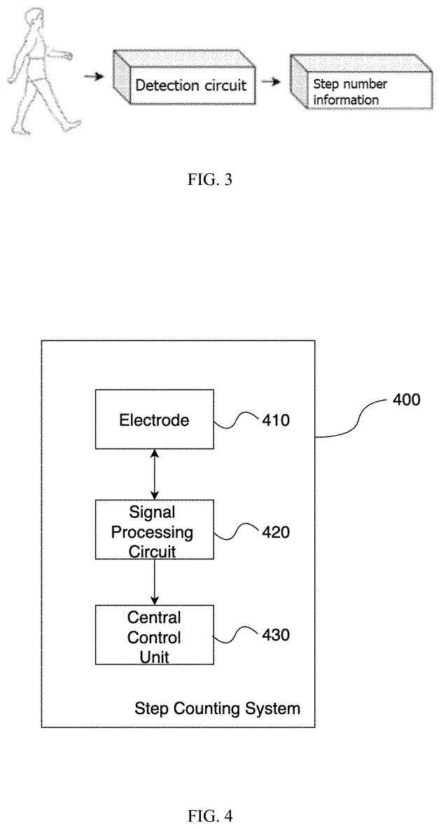

FIG. 3 is the basic working steps of a step counting system provided by an embodiment of the present disclosure:

Step 1: A detection circuit directly or indirectly detects the human body capacitance variation when walking. The detection circuit can also directly or indirectly catch the variation of other physical properties caused by human body capacitance variation, such as charges on an electrode that is in contact or coupled with the human body.

Step 2: A central control unit performs numerical analysis on the signal output of the detection circuit to obtain step number information.

As shown in FIG. 4: FIG. 4 is a schematic structural diagram of a step counting system provided by an embodiment of the present disclosure. In this embodiment, a step counting system 400 comprises an electrode 410, a signal processing circuit 420, and a central control unit 430. The electrode 410 is electrically connected to the signal processing circuit 420, and the signal processing circuit 420 is electrically connected to the central control unit 430. The electrode 410 is used for acquiring an electric signal caused by the variation of the human body capacitance. When the electrode 410 is in direct contact with the human body, the electrical signal on the electrode 410 caused by the human body capacitance variation is the conduction current signal; when the electrode 410 is coupled with the human body, the electrical signal on the electrode 410 caused by the human body capacitance variation is the displacement current. The signal processing circuit 420 provides a bias voltage for the electrode 410 and has an input impedance higher than one kiloohm. This bias voltage also acts as a charge source for the electrode. Because the human body capacitance is only about one hundred picofarads, the human body capacitance variation caused by human walking is extremely weak. To sense the weak human body capacitance variation, the step counting system converts the sensing of the slight human body capacitance variation into the sensing of the weak charge flow at the electrode caused by the human body capacitance variation. The signal processing circuit 420 performs signal processing to the electrical signals on the electrode 410 and transmits the processed signals to the central control unit 430. The central control unit 430 analyzes the signal through a corresponding step counting algorithm to obtain step count information.

According to some embodiments, the electrode is in direct contact or coupled with the human body. When the human body lifts or lands a foot, the human body capacitance changes, and the change causes the flow of charges on the body, which also manifests as the flow of charges at the electrode.

According to some embodiments, the signal processing circuit provides a bias voltage to the electrode and has an input impedance higher than one kiloohm. Therefore, the flow of charges on the electrode can be reflected as the observable and readable voltage change on the electrode. The signal processing circuit performs signal processing to the voltage signal.

According to some embodiments, the signal processing circuit includes, but is not limited to, one or more of the following circuit units: filtering unit, amplification unit, and analog-to-digital conversion unit.

Advantageously, by placing a capacitor between the electrode and the signal processing circuit, the amplitude and response time of the voltage signal variation can be adjusted; thus, the accuracy of the step number detection can be further improved.

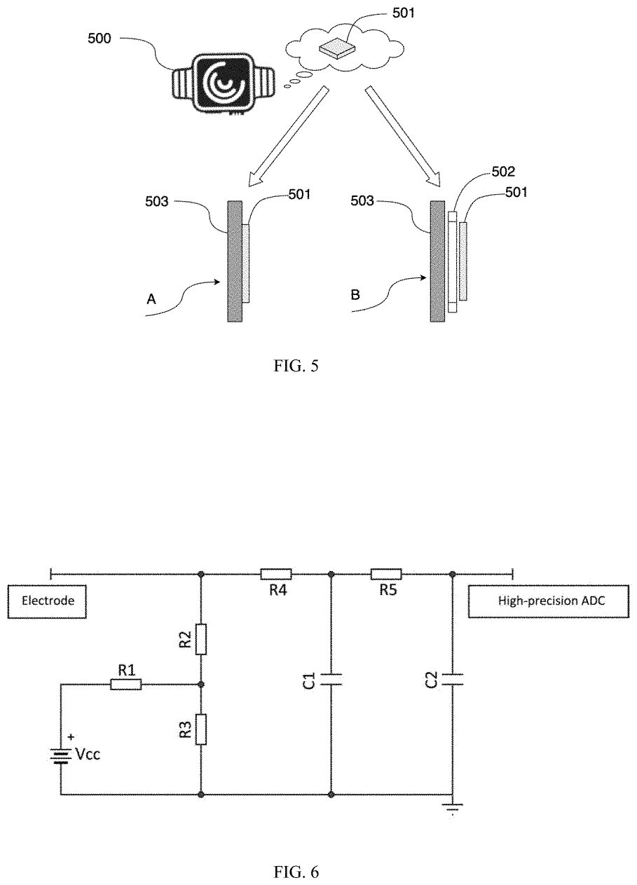

As shown in FIG. 5, FIG. 5 is a schematic diagram of a connection mode provided by an embodiment of the present disclosure.

In this embodiment, as shown in case of A in FIG. 5, the electrode 501 placed in the smart wearable device 500 is in direct contact with the skin 503, and the electrical signal on the electrode caused by the human body capacitance variation is the conduction current signal; As shown in case of B in FIG. 5, the electrode 501 is coupled with the skin 503, that is, a medium 502 is sandwiched between the electrode 501 and the skin 503, in this case, the electrical signal on the electrode caused by the human body capacitance variation is the displacement current signal.

According to some embodiments, the medium 502 mentioned above is a low-conductivity medium, including but not limited to air, fabric, or rubber wristbands.

According to some embodiments, the material, size, and placement position of the electrode 501 are not limited.

Advantageously, the electrode 501 is a piece of conductor inside or on the surface of the smart wearable device 500.

As shown in FIG. 6: FIG. 6 is a circuit diagram of a signal processing circuit provided by an embodiment of the present disclosure. In this embodiment, the electrode is connected to a resistor network composed of R1, R2, and R3. This network provides a stable bias voltage for the electrode and an input impedance higher than one kiloohm for the signal processing circuit. Providing a stable bias voltage can give the electrode the potential to have the flowable charges and a observable and readable voltage. R4, C1, R5, and C2 form a second-order low-pass filter network. The output of the circuit is connected to a high-precision analog-to-digital conversion module to ensure that the output voltage variation caused by the charge flow at the electrode is detectable.

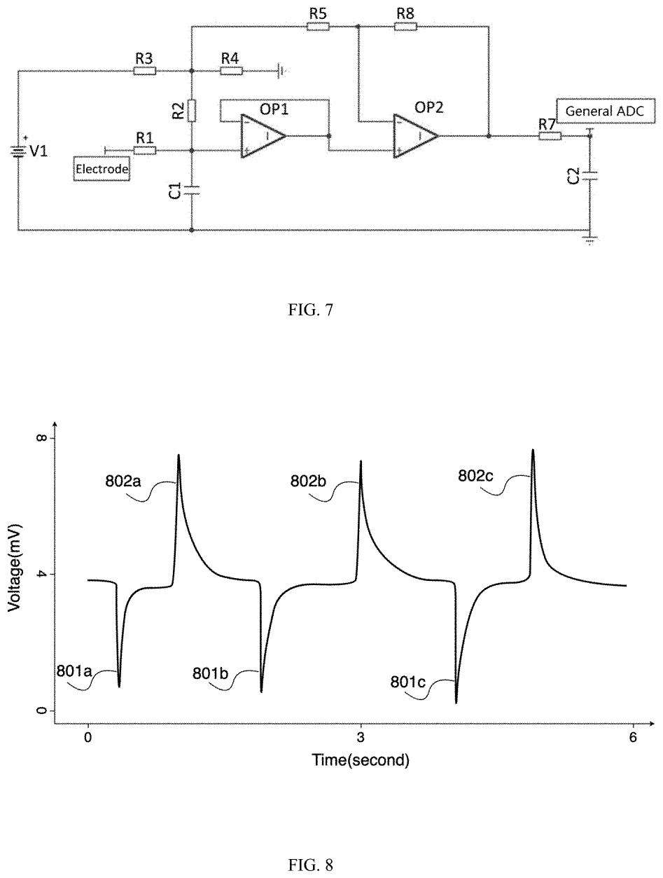

As shown in FIG. 7, FIG. 7 is a circuit diagram of a signal processing circuit provided by another embodiment of the present disclosure. In this embodiment, a general analog-to-digital conversion module replaces the high-precision analog-to-digital conversion module, as shown in FIG. 6, but the operational amplifiers are added. The amplification circuit is composed of operational amplifier OP1 and operational amplifier OP2. Resistors R2, R3, and R4 form a resistor network to provide a stable bias voltage for the electrode and an input impedance higher than one kilo-ohm for the signal processing circuit. Providing a stable bias voltage can give the electrode the potential to have the flowable charges and a observable and readable voltage. R1, C1, R7, and C2 are low-pass filter networks. The circuit design can reduce the cost, power consumption, and volume of the signal processing circuit. The circuit uses the operational amplifier to amplify the weak voltage variation caused by the charge flow at the electrode. A general analog-to-digital conversion module can detect the voltage variation at the output caused by the charge flow at the electrode.

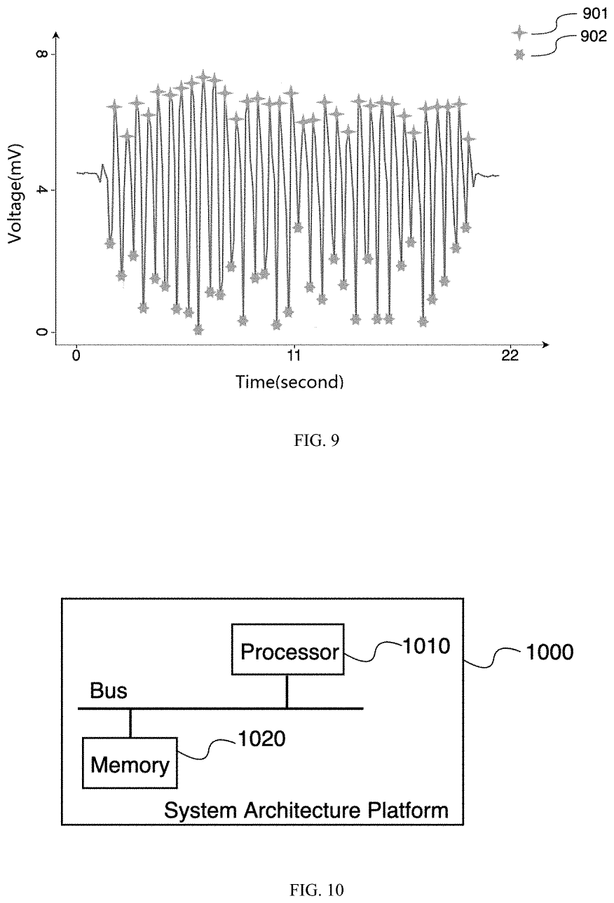

FIG. 8 and FIG. 9 show the detected signals of foot-lifting and foot-landing when the circuit shown in FIG. 6 is used as the signal processing circuit and the step counting system shown in FIG. 4 is worn on the wrist with the electrode in contact with the skin. When lifting or landing the foot, the human body capacitance varies due to the distance change between the human body and the ground. The human body capacitance variation is expressed as the process of charge flow at the electrode, that is, a charging and discharging process. And the variation is expressed as voltage variation at the output of the signal processing circuit. The voltage signal has peaks in different directions. When walking at a slow speed, the peak voltage signals 801a, 801b, 801c are obtained by the central control unit when the foot lifts, and the peak voltage signals 802a, 802b, 802c are obtained by the central control unit when the foot lands. When the human body walks faster and the electrode contacts the skin, the collected voltage signal is shown in FIG. 9. The peak signal 902 is the peak voltage signal obtained by the central control unit when the foot lifts, and 901 is the peak voltage signal obtained by the central control unit when the foot lands. Since the time interval between the foot-lifting and foot-landing is relatively short, the voltage at the output of the signal processing circuit has not completely dropped back to the bias voltage level, and the next peak signal occurs. The frequency of peaks represents the frequency of foot-lifting and foot-landing.

According to some embodiments, the direction of the peak signal is the opposite of the above description of FIG. 8 and FIG. 9. When a foot-lifting happens, the peak signal occurs as 802a, 802b, 802c in FIGS. 8 and 901 in FIG. 9, and when a foot-landing happens, the peak signal occurs as 801a, 801b, 801c in FIGS. 8 and 902 in FIG. 9. The direction of the peak signal is depending on factors like the connection mode of the electrode.

As shown in FIG. 10, FIG. 10 is a schematic diagram of a system architecture platform for executing a step counting method provided by an embodiment of the present disclosure.

The system architecture platform 1000 of the embodiment of the present disclosure comprises one or more processors 1010 and a memory 1020, and one processor 1010 and one memory 1020 are taken as examples in FIG. 10.

The processor 1010 and the memory 1020 may be connected by a bus or other ways, and connecting by bus is taken as an example in FIG. 10.

As a non-transient computer-readable storage medium, the memory 1020 may be used to store non-transient software programs and non-transient computer-executable programs. In addition, the memory 1020 may comprise a high-speed random access memory, and may also comprise a non-transitory memory, such as at least one disk memory device, a flash memory device, or other non-transitory solid storage devices. In some embodiments, the memory 1020 optionally comprises a memory 1020 remotely disposed with respect to the processor 1010, which may be connected to the system architecture platform 1000 through a network. Examples of the networks above comprise, but are not limited to, the Internet, intranet, local area networks, mobile communication networks, and combinations thereof.

Those skilled in the art can understand that the apparatus structure shown in FIG. 10 does not constitute a limitation to the system architecture platform 1000, and may comprise more or less components than the illustrated components, or combine some components, or have different component arrangements.

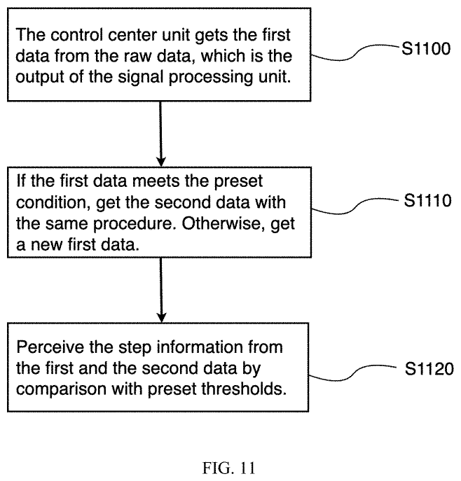

As shown in FIG. 11, FIG. 11 is a basic flowchart of a step counting algorithm provided by an embodiment of the present disclosure. The step counting algorithm of the embodiment of the present disclosure comprises, but is not limited to, step S1100, step S1110, and step S1120.

At step S1100, The control center unit gets the first data from the raw data, which is the output of the signal processing unit.

At step S1110, If the first data meets the preset condition, get the second data with the same procedure. Otherwise, get a new first data.

At step S1120, Perceive the step information from the first and the second data by comparisons with preset thresholds.

In this embodiment, as the distance between the human body and the ground changes when the human body performs a foot-lifting or foot-landing action, resulting in the human body capacitance variation, an electric signal caused by the human body capacitance variation on the electrode that is in contact or coupled with the human body occurs. Then the signal processing is carried out on the electric signal and the central control unit gets the first data from the raw data of the processed electric signal; when the first data meets the preset condition, the second data is acquired with the same procedure. It is also necessary to confirm whether the second data meets the preset condition. Afterwards, the step number information is obtained according to the comparison of the first data and the second data with predefined thresholds. Judging whether the acquired data meets the preset condition can filter out some signals of human body capacitance changes caused by non-foot-lifting or non-foot-landing action of the human body.

According to some embodiments, the first data and the second data include but are not limited to one or more of the following data: a single raw data acquired at the central control unit, a group of raw data acquired at the central control unit, a single change rate data of the raw data acquired at the central control unit, a group change rate data of raw data acquired at the central control unit, a single rate of change rate data of the raw data acquired at the central control unit, a group rate of change rate data of raw data acquired at the central control unit.

According to some embodiments, the preset conditions include but are not limited to one or more of the following comparison: bigger than a threshold, smaller than a threshold, bigger than or equal to a threshold, smaller than or equal to a threshold.

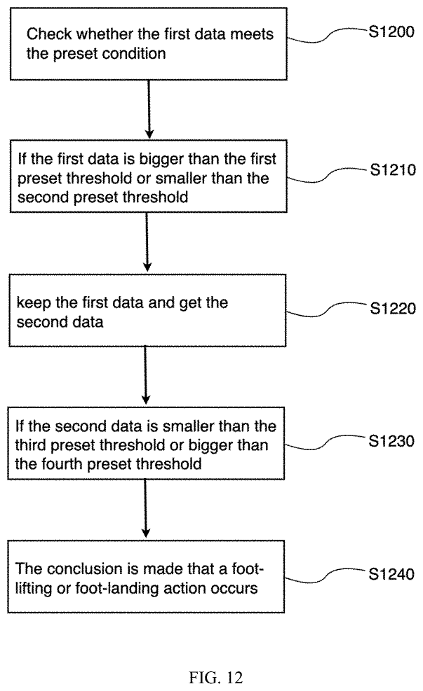

FIG. 12 is a flowchart of a part of the step counting algorithm provided by an embodiment of the present disclosure; This part of step counting algorithm of the embodiment of the present disclosure comprises, but is not limited to, step S1200, step S1210, step S1220, step 1230, and step 1240.

At step S1200, check whether a first data meets a preset condition.

At step S1210, when the result shows that the first data is bigger than the first preset threshold or smaller than a second preset threshold.

At step S1220, keep the first data and acquire a second data.

At step S1230, when the result shows that the second data is smaller than the third preset threshold or bigger than the fourth preset threshold.

At step S1240, it is concluded that a foot-lifting action or a foot-landing action occurs.

According to some embodiments, the related thresholds value can be equal.

According to some embodiments, the first data and the second data include but are not limited to one or more of the following data: a single raw data acquired at the central control unit, a group of raw data acquired at the central control unit, a single change rate data of the raw data acquired at the central control unit, a group change rate data of raw data acquired at the central control unit, a single rate of change rate data of the raw data acquired at the central control unit, a group rate of change rate data of raw data acquired at the central control unit.

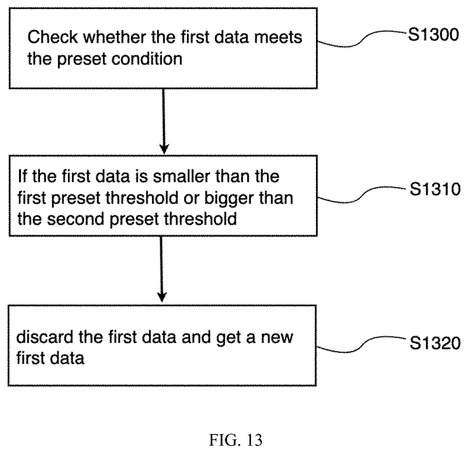

FIG. 13 is a flowchart of a part of the step counting algorithm provided by an embodiment of the present disclosure; This part of step counting algorithm of the embodiment of the present disclosure comprises, but is not limited to, step S1300, step S1310, and step S1320.

At step S1300, check whether a first data meets a preset condition.

At step S1310, when the result shows that the first data is smaller than or equal to the first preset threshold and bigger than or equal to a second preset threshold.

At step S1320, discard the first data and acquire a new first data.

According to some embodiments, the first data and the second data include but are not limited to one or more of the following data: a single raw data acquired at the central control unit, a group of raw data acquired at the central control unit, a single change rate data of the raw data acquired at the central control unit, a group change rate data of raw data acquired at the central control unit, a single rate of change rate data of the raw data acquired at the central control unit, a group rate of change rate data of raw data acquired at the central control unit.

In addition, an embodiment of the present disclosure further provides a computer-readable storage medium, wherein the computer-readable storage medium stores a computer-executable instruction, and the computer-executable instruction is executed by one processor or controller, for example, by one processor in the controller embodiment above, which can cause the processor to execute the step counting algorithm in the foregoing embodiments, for example, to execute the above-described algorithm steps S1100 to S1120 in FIG. 11, algorithm steps S1200 and S1240 in FIG. 12, algorithm steps S1300 to S1320 in FIG. 13.

Those of ordinary skills in the art will appreciate that all or some of the steps and systems in the methods disclosed above may be implemented as software, firmware, hardware, and suitable combinations thereof. Some physical components or all of the physical components may be implemented as software executed by a processor, such as a central processing unit, a digital signal processor or a microprocessor, or implemented as hardware, or implemented as an integrated circuit, such as an application specific integrated circuit. Such software may be distributed on a computer-readable medium, which may include a computer storage medium (or non-transitory medium) and a communication medium (or transitory medium). As well known to those of ordinary skills in the art, the term computer storage medium includes volatile and non-volatile, removable and non-removable media implemented in any method or art for storing information (such as computer-readable instruction, data structure, programming module or other data). The computer storage medium includes, but is not limited to, RAM, ROM, EEPROM, flash memory or other memory technology, CD-ROM, digital versatile disc (DVD) or other optical disc memory, magnetic cassette, magnetic tape, magnetic disk memory or other magnetic memory device, or may be any other medium that can be used to store the desired information and can be accessed by a computer. Moreover, it is well known to those of ordinary skills in the art that the communication medium typically includes computer-readable instructions, data structures, program modules, or other data in a modulated data signal, such as a carrier wave or other transmission mechanism, and may include any information delivery medium.

The foregoing describes the preferred embodiments of the present disclosure in detail, but the embodiments of the present disclosure are not limited to the foregoing embodiments. Those skilled in the art can make various equal deformations or replacements without departing from the spirit of the embodiments of the present disclosure, and these equal deformations or replacements shall all fall within the scope limited by the claims of the embodiments of the present disclosure.

What is claimed is:

1. A step counting method, comprising:
acquiring, by an electrode that is in contact or coupled with a human skin, an electric signal caused by a human capacitance variation during foot-lifting and foot-landing of a human body; and
processing, by a signal processing circuit electrically connected with the electrode, the electric signal, and outputting a signal stream that records the foot-lifting and foot-landing information of the human body to a central control unit; and
analyzing, by the central control unit electrically connected with the signal processing circuit, the received signal stream, counting the foot-lifting and foot-landing information of the human body recorded in the signal stream.
wherein the signal processing circuit provides a bias voltage for the electrode, and the signal processing circuit has an input impedance larger than one kilo-ohm.

2. The step-counting method according to claim 1, wherein the signal processing circuit supplies charges for the electrode as a charge source.

3. The step-counting method according to claim 1, wherein the human body capacitance describes the electric field between the human body and the environment.

4. The step-counting method according to claim 1, wherein the step number information is acquired by the following steps:
a) The signal processing circuit acquires the electrical signal caused by variations in the human body capacitance from the electrode in contact or coupled with the skin and forwards the processed signal to the central control unit for the first data.
b) When the first data meets the preset condition, the central control unit acquires the second data and checks if the second data meets the preset condition. Then, the step number is obtained according to the first and second data.

5. The step-counting method according to claim 4, wherein the first data and the second data include but are not limited to one or more of the following data: a single raw data, a group of raw data, a single change rate data of the raw data, a group change rate data of raw data, a single rate of change rate data of the raw data, a group rate of change rate data of raw data.

6. The step-counting method according to claim 4, wherein the preset condition is a threshold value based on the raw data or the change rate of raw data or the rate of change rate of raw data for comparison.

7. A step counting system based on human body capacitance, comprising:
An electrode, which is in contact or coupled with the human skin; and
a signal processing circuit that is electrically connected with the electrode to process the electric signal on the electrode and outputs a signal stream that records foot-lifting and foot-landing information; and
a central control unit that is electrically connected with the signal processing circuit, analyzes the signal stream received from the signal processing circuit and counts the foot-lifting or foot-landing number.
wherein, during the foot-lifting or foot-landing action, the human body capacitance changes, a conduction current or displacement current signal occurs on the electrode, and after the current signal being processed by the signal processing circuit, a signal is received by the central control unit which records the step information.
wherein the signal processing circuit provides a bias voltage for the electrode, and the signal processing circuit has an input impedance larger than one kiloohm.

8. The step-counting system according to claim 7, wherein the signal processing circuit, as a charge source, supplies charges for the electrode.

9. The step counting system according to claim 7, wherein the signal processing circuit includes, but is not limited to, one or more of the following functions: filtering, amplification, analog-to-digital conversion.

10. The step counting system according to claim 7, wherein the output signal from the signal processing circuit carries the step information, and the central control unit obtains the step number by detecting and counting the peaks in the received signal.

11. The step counting system according to claim 7, wherein all or part of components or units in the step counting system are integrated into a single silicon chip.

12. A computer-readable storage medium, wherein the computer-readable storage medium stores computer-executable instructions, and the computer-executable instructions are configured to make a computer execute the step-counting method according to claim 1.
